(19) United States
(12) Patent Application Publication
(10) Pub. No.: US 2014/0371254 A1
(43) Pub. Date: Dec. 18, 2014

(54) NOVEL TREATMENT OF GEFITINIB-RESISTANT NON-SMALL-CELL LUNG CANCER

(71) Applicant: Macau University of Science and Technology, Macau (CN)

(72) Inventors: Lai Han LEUNG, Macau (CN); Xing Xing FAN, Macau (CN); Liang LIU, Macau (CN)

(21) Appl. No.: 14/285,651

(22) Filed: May 23, 2014

Related U.S. Application Data
(60) Provisional application No. 61/835,642, filed on Jun. 17, 2013.

(51) Int. Cl. A61K 31/4741

(52) U.S. Cl. CPC A61K 31/4741
USPC 514/279

(57) ABSTRACT

The present invention discloses a method of preventing and treating Gefitinib-resistant non-small-cell lung cancer, comprising administering an effective amount of an alkaloid. A pharmaceutical composition comprising an alkaloid admixed with a pharmaceutical carrier for treating Gefitinib-resistant non-small-cell lung cancer is also disclosed therein.

### CROSS-REFERENCE TO RELATED APPLICATION

This application claims benefit under 35 U.S.C. §119(e) of U.S. Provisional Application having Ser. No. 61/835,642 filed 17 Jun. 2013, which is hereby incorporated by reference herein in its entirety.

### FIELD OF INVENTION

This invention relates to a novel treatment for treating Gefitinib-resistant non-small-cell lung cancer, in particular, a novel treatment involving the use of alkaloid.

### BACKGROUND OF INVENTION

Lung cancer is the leading cause of cancer deaths globally [1]. Non-small-cell lung cancer (NSCLC) accounts for over 80% of all the histological classified lung cancer cases, and patients are often diagnosed at the advanced stages of the disease; therefore the prognosis of lung cancer remains poor [2]. With the advanced development of DNA sequencing technology, the therapeutic strategy of NSCLC has been modified towards personalized therapy. Some specific driver genetic mutations have been identified in NSCLC, such as Epidermal Growth Factor Receptor (EGFR) [3, 4], EML4-ALK fusion gene [5] and ROS fusion gene [6], which direct the development of molecular-targeted drug discovery to target theses mutations. Among those mutations, EGFR mutation is the most frequently observed gene mutation in the Eastern Oriental population, especially in subgroup of patients who are non-smoker, female, clinically diagnosed with adenocarcinoma and early onset. The common activating mutation of EGFR is a substitution mutation of EGFR^{L858R}, which makes EGFR constitutively activated even without EGF stimulation, resulting in downstream activation of anti-apoptotic signaling. Gefitinib, which is a tyrosine kinase inhibitor (TKI), can specifically inhibit EGFR as well as its downstream survival signaling pathway [7]. However, despite the initial significant responses to Gefitinib treatment, like other chemotherapeutic agents, patients acquire resistance to Gefitinib ultimately, and the median time to disease progression is just about 12 months [8]. The most common reason of Gefitinib resistance is the presence of additional EGFR mutation (EGFR^{L858R+T790M}), which accounts for over 49% of all the resistance cases. The additional T790M mutation will provide steric hindrance to TKI due to the bulkiness of methionine (M), and thus the overall pharmaceutical effect of Gefitinib is weakened.

Therefore, there is an urgent need to identify EGFR crosstalk pathways and to discover more effective agents as new candidate drugs for Gefitinib-resistant NSCLC patients.

### SUMMARY OF INVENTION

In the light of the foregoing background, it is an object of the present invention to provide an alternate treatment for Gefitinib-resistant non-small-cell lung cancer.

In the first aspect, the present invention relates to a method of preventing and treating Gefitinib-resistant non-small-cell lung cancer, comprising administering an effective amount of an isoquinoline alkaloid.

In one embodiment, the isoquinoline alkaloid is sanguinarine.

In another aspect of this invention, the present invention provides a pharmaceutical composition comprising an isoquinoline alkaloid admixed with a pharmaceutical carrier for treating Gefitinib-resistant non-small-cell lung cancer.

In one embodiment, the isoquinoline alkaloid is sanguinarine.

In another aspect of this invention, a dietary supplement comprising an isoquinoline alkaloid admixed with a pharmaceutical carrier for treating Gefitinib-resistant non-small-cell lung cancer is provided.

In one embodiment the isoquinoline alkaloid is sanguinarine.

### BRIEF DESCRIPTION OF FIGURES

FIGS. 1a and 1b show EGFR degradation induced by sanguinarine.

FIGS. 2a and 2b show the induced apoptosis by sanguinarine in Gefitinib-resistant NSCLC cell line H1975.

FIG. 3 shows the induced apoptosis by sanguinarine in H1975 cells via mitochondria dependent pathway.

FIGS. 4a and 4b show the induced apoptosis by sanguinarine in H1975 in caspases-dependent manner.

FIGS. 5a, 5b and 6 show the effect of reactive oxygen species (ROS) generation in sanguinarine-induced apoptosis in H1975 cells.

### DETAILED DESCRIPTION OF THE PREFERRED EMBODIMENTS

Sanguinarine, which is a natural benzophenanthridine alkaloid, is isolated from Chinese medicinal herb Macleaya cordata and also from North American herb sanguinaria canadensis. As the major constituent, the pharmacological effects of sanguinarine have been widely studied in many fields for a long time, for example, as an anti-microbe agent [9, 10], an anti-inflammation agent [11, 12] and an anti-oxidation agent [13, 14]. It is also approved by the FDA as an antibacterial or antiplaque agent in toothpastes in 2003 [15]. However, its effect on NSCLC and especially on Gefitinib-resistant NSCLC has never been studied and thus not readily known to one skilled in the art. In the instant invention, it is the first time to report the anticancer effect of sanguinarine on Gefitinib-resistant NSCLC, which may provide hopes to cancer patients who have developed Gefitinib resistance.

### EXAMPLE 1

### Materials and Methods Used in the Studies of This Invention

### Chemicals

Sanguinarine powder was purchased from Sigma Aldrich. Antibodies of Bcl-2, Bax and Bcl-xl were purchased from Santa Cruz Biotechnology while antibodies of phospho-P38, phosphor-JNK, phospho-AKT, Total-EGFR, phosphor-EGFR Y1045 were purchased from Cell Signaling Technology.

### Cell Lines and Cell Culture

Four NSCLC cell lines, H1975 (EGFR^{L858R+T790M}), H1650 (EGFR^{Exon19 deletion}), H2228 (EML4-ALK fusion gene) and A549 (EGFR^{Wild type}) were purchased from ATCC. All cell lines were cultivated with RPMI 1640 medium supplemented with 10% fetal bovine serum (Gibco), 100 u/ml penicillin and 100 μg/ml streptomycin (Gibco). All cells were cultivated at 37° C. in an incubator supplying with 5% CO₂.

### 3-(4,5-Dimethylthiazol-2-yl)-2,5-diphenyltetrazolium bromide (MIT) assay

NSCLC cells were seeded on a 96-well microplate at a confluence of 4000 cells/well, and were cultured overnight to allow cell adhesion. Wide concentration of range of sanguinarine was added into the wells and the plate was incubated for 24 or 48 hrs with vehicle (DMSO) as the control. Each dosage was repeated in triplicate. 10 μl of MTT (5 mg/ml) was added to each well and the plate was placed back into the incubator for another 4 hrs to allow the entry of MTT into the cells. Then 100 μl of the resolved solution (10% SDS and 0.01% M HCL) was added to each well and the plate was further incubated at 37° C. for 4 hrs until the formazan crystals had dissolved. Finally, the absorbance of the plate was measured at 570 nm (absorbance) and 650 nm (reference) by a microplate reader (Tecan). The percentage of cell viability is calculated as the percentage change of the absorbance of treated cells to the untreated cells.

### Assessment of Apoptosis Levels by Annexin V/PI Staining with Flow Cytometry

Annexin V/PI staining is used to detect early and late stages of apoptosis. According to the manufacturer's protocol (BD Pharmingen), 5×10⁵ cells of the treated and control groups were harvested, washed, and double-stained with Annexin V/PI for 15 min at room temperature in dark. Then apoptotic cells were quantitatively counted by a flow cytometer (BD FACSAria III). Early stage of apoptotic cells were stained with Annexin V-positive and PI-negative, while the late stage of apoptotic cells were stained with Annexin V-positive and PI-positive.

### Western Blot Analysis

Cells were lysed in RIPA lysis buffer with protease and phosphatase inhibitors added for total whole cell protein extraction. The concentration of the total protein extract was determined by Bio-Rad DCTM protein assay kit (Bio-Rad). Then samples were loaded and separated onto a 10% SDS-PAGE gel and then the separated proteins were transferred to a Nitrocellulose (NC) membrane. Membranes were blocked with 5% milk without fat in TBST for 1 hour at room temperature. Primary antibodies (1:1000 dilutions for Cell Signaling antibodies; 1:500 dilutions for Santa Cruz antibodies) were added and incubated overnight at 4° C. On the other day, membrane was washed by TBST for three times (5 mins/wash), while secondary fluorescent antibody (1:10000 dilutions) was added to membrane and incubated at room temperature for 1 hr. Actin was used as loading control and for normalization. The signal intensity of the membranes was detected by Odessy (LI-COR).

### Detection of ROS Generation

Levels of reactive oxygen species (ROS) generation was analyzed by using fluorescent probe dichlorofluorescein diacetate (DCFDA) staining, which is a specific superoxide tracing dye. Cells were pretreated with DCFDA at working concentration of 20 μM (Abcam) for 30 min at 37° C. prior to sanguinarine treatment. Then cells were incubated with sanguinarine and vehicle control for 30 mins, 1 hr and 2 hrs, and then were harvested and re-suspended in PBS. Fluorescence signal was measured with flow cytometer with excitation and emission settings of 488 and 525 nm, respectively.

### Statistical Analysis

All of the data is expressed as mean±SEM of three individual experiments. Differences between groups were determined by one way analysis of variance (ANOVA), with p values<0.05 was considered as significant.

### EXAMPLE 2

### Study of Sanguinarine Against Four NSCLC Cell Lines After MTT Cytotoxicity Assay

This example described the cytotoxicity effect induced by sanguinarine in four NSCLC cell lines utilizing MTT cytotoxicity assay. Among the four cell lines, A549 is wild type EGFR, while the other three are Gefitinib-resistant NSCLC lines with different mutations. H2228 contains EML4-ALK fusion gene mutation and could be used as EGFR survival independent control cells. IC₅₀ values of each cell line after a 24 hr treatment are shown in Table 1.

TABLE 1

| | IC₅₀ value | IC₅₀ value | |
|---|---|---|---|
| | after 24 hrs | after 48 hrs | |
| | Treatment | Treatment | Gefitinib |
| Cell lines | (μM) | (μM) | Sensitivity |
| H1975 (EGFR^{L858R+T790M}) | 0.68 ± 0.24 | 0.58 ± 0.14 | Resistant |
| H1650 (EGFR^{Exon19 deletion}) | 3.47 ± 1.31 | 2.64 ± 1.31 | Resistant |
| H2228 (EML4-ALK | 3.73 ± 1.23 | 3.08 ± 1.56 | Resistant |
| fusion gene) | | | |
| A549 (EGFR^{Wild type}) | 5.71 ± 1.73 | 3.83 ± 1.11 | Moderately |
| | | | sensitive |

Conclusion: From the results shown in Table 1, sanguinarine has cytotoxicity effect in human NSCLC cell lines, especially in H1975 (EGFR^{L858R+T790M}); while it shows relatively low cytotoxicity effect in A549 (EGFR^{Wild type}) cell lines. Thus, sanguinarine is shown to be effective in treating Gefitinib-resistant non-small-cell lung cancer.

### EXAMPLE 3

### Western Blot Analysis of the Protein Level of EGFR After Sanguinarine Treatment

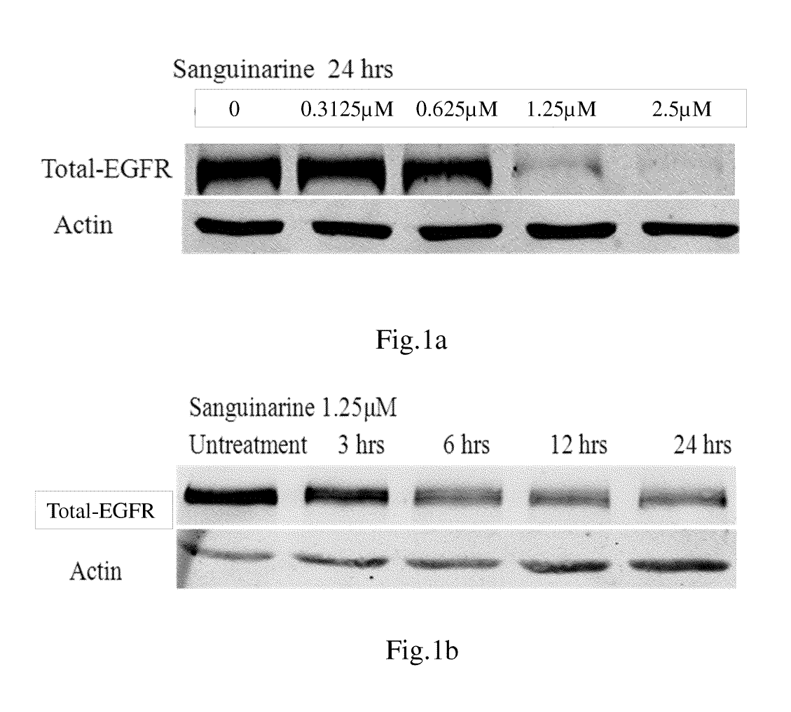

In Example 3, the protein level of EGFR after sanguinarine treatment with difference dosage and duration was analyzed. FIG. 1a shows the results of western blot analysis of the protein level of EGFR after treating with sanguinarine of different concentrations for 24 hrs. FIG. 1b shows the results of western blot analysis of the protein level of EGFR after treating with sanguinarine at 1.25 μM for 0-24 hrs.

Conclusion: The results indicate that sanguinarine induced EGFR degrades in a dose-dependent and time-dependent manner.

### EXAMPLE 4

### Study of Sanguinarine-Induced Apoptosis in Gefitinib-Resistant NSCLC Cell Line H1975

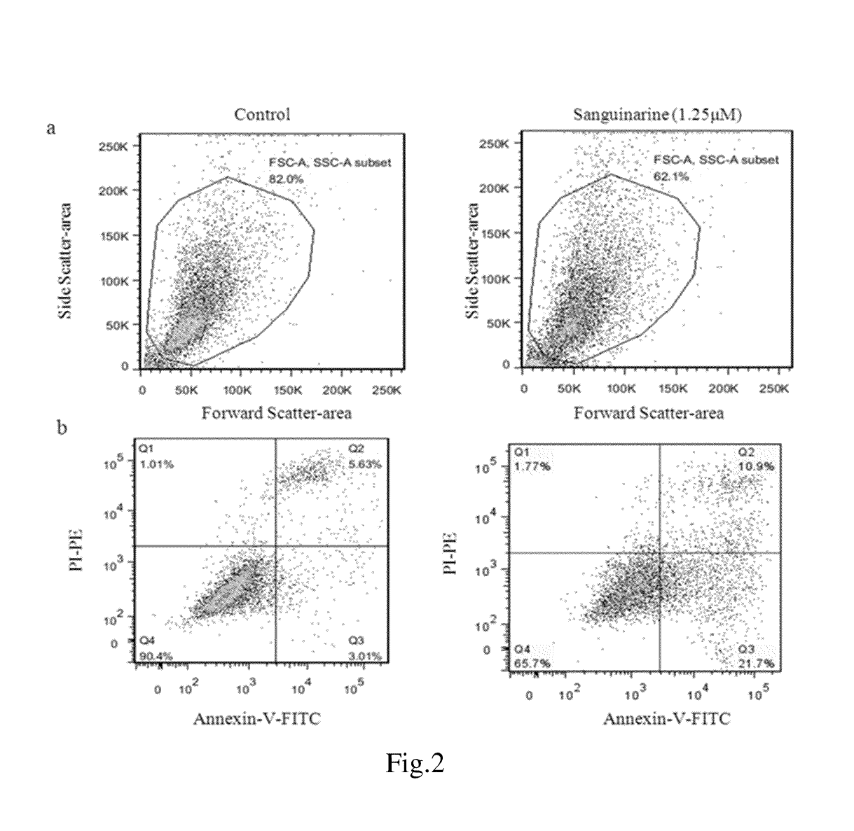

Example 4 further studies the sanguinarine-induced apoptosis in Gefitinib-resistant NSCLC cell line H1975 (EGFR^{L858R+T790M}) Upon treatment with sanguinarine at a concentration of 1.25 μM for 24 hrs, significant induction of apoptosis in H1975 cells was observed, as shown in FIG. 2.

As shown in FIG. 2a, intact cells were gated in the same area, and the treatment of sanguinarine resulted in 20% more cell debris. In FIG. 2b, the percentages of both early stage (Q3) and late stage (Q2) apoptotic cells were quantitatively counted by flow cytometer, and the result shows that there is a significant 7.2-fold increase in early stage of apoptosis of the sanguinarine-treatment group as compared with the control group.

Conclusion: The results indicate that sanguinarine induces apoptosis in Gefitinib-resistant NSCLC cell line H1975 significantly.

### EXAMPLE 5

### Study of Sanguinarine-Induced Apoptosis in H1975 Cells via Mitochondria Dependent Pathway

Example 5 analyzed the protein level of Bcl-2, Bcl-xl and Bax after sanguinarine treatment with difference dosages for 24 hours.

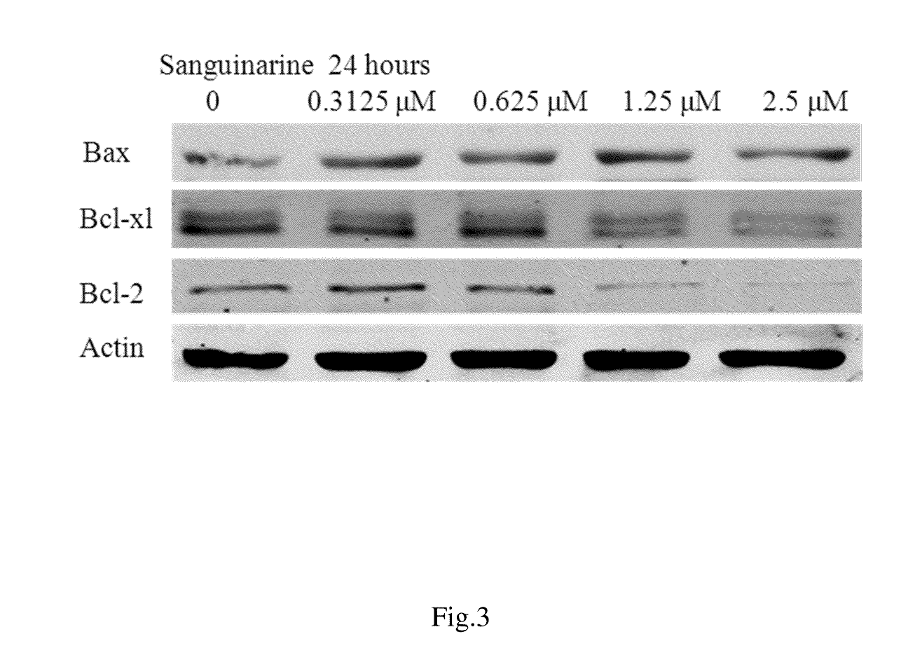

Conclusion: From the results shown in FIG. 3, sanguinarine is shown to remarkably suppress the expression of Bcl-2, Bcl-xl, and slightly up-regulate Bax, which will up-regulate the ratio of Bax/Bcl-2, leading to the disruption of mitochondrial membrane integrity and further leading to significant apoptosis in Gefitinib-resistant NSCLC cell line H1975.

### EXAMPLE 6

### Study of Sanguinarine-Induced Apoptosis in H1975 in Caspases-Dependent Manner

Example 6 further studies if the sanguinarine-induced apoptosis in H1975 (EGFR^{L858R+T790M}) is in caspases-dependent manner H1975 cells were treated with 1.25 μM sanguinarine and 1.25 μM sanguinarine together with 50 μM pan-caspase inhibitor for 24 hrs.

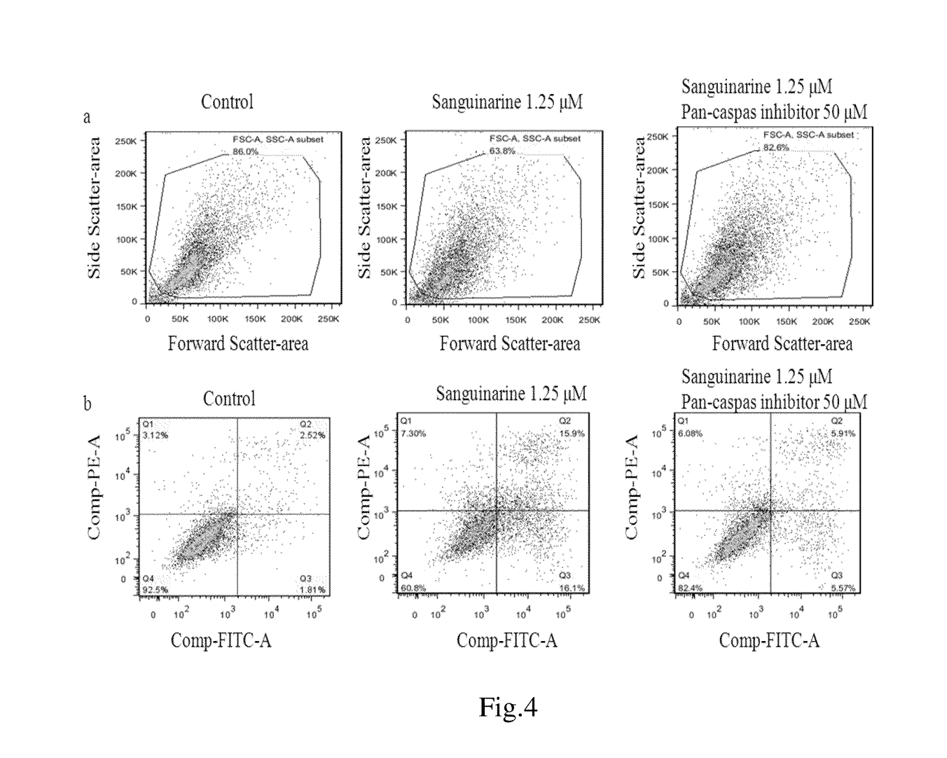

As shown in FIG. 4a, intact cells were gated in the same area, and the treatment of 1.25 μM sanguinarine together with 50 μM pan-caspase inhibitor resulted in 20% less cell debris compared with the group treated with 1.25 μM sanguinarine only. As shown in FIG. 4b, the percentages of both early stage (Q3) and late stage (Q2) apoptotic cells were quantitatively counted by flow cytometer, and the result shows that the percentages were decreased about 3-fold in both early and late stages of apoptosis of the sanguinarine & pan-caspase inhibitor treatment group compared with the sanguinarine group.

Conclusion: The results show that pan-caspase inhibitors can attenuate sanguinarine-induced apoptosis in H1975 cells; in other words, caspases activation is necessary for the apoptosis induced by sanguinarine.

### EXAMPLE 7

### Study on Reactive Oxygen Species (ROS) Generation in Sanguinarine-Induced Apoptosis in H1975 Cells

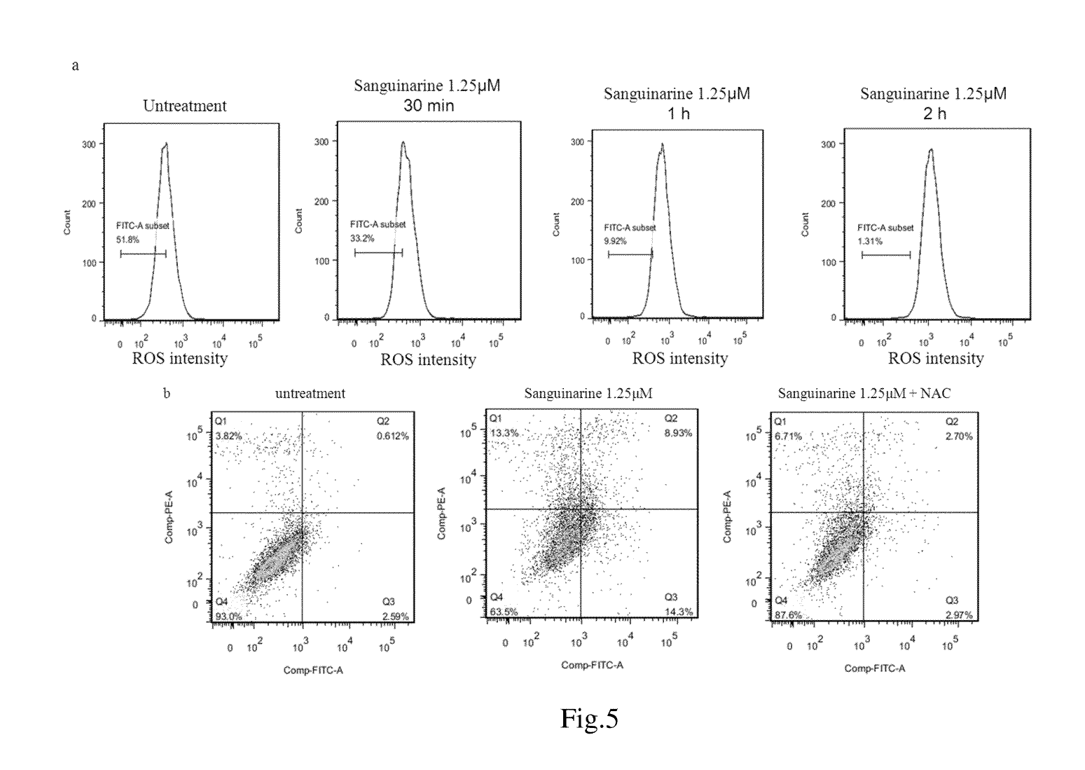

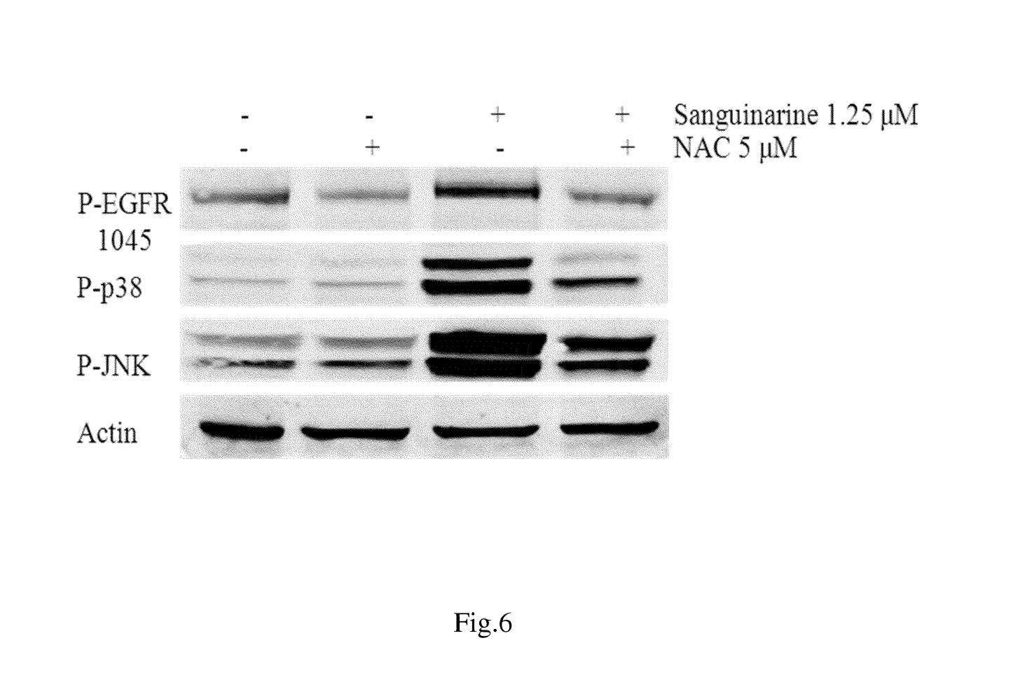

At 30 minutes after 1.25 μM sanguinarine treatment, ROS was greatly accumulated and the ROS intensity was shown in FIG. 5a. ROS intensity was increased with the increase of sanguinarine treatment time. Sanguinarine-induced apoptosis in H1975 cells, in which the cells were pre-treated with 5 μM NAC and a ROS generation inhibitor, was also studied. As shown in FIG. 5b, it can be observed that apoptosis induced by sanguinarine was completely blocked. Further, the protein levels of JNK, p38 and EGFR after the treatment of 5 μM NAC and/or 1.25 μM sanguinarine were shown in FIG. 6, indicating that the ROS generation inhibitor can also block the phosphorylation of JNK, p38 and EGFR degradation. Also, P-EGFR 1045 as shown in FIG. 6 is a tyrosine(Y) phosphorylation site of EGFR at amino acid position 1045 (Y1045) and phosphorylation of this site will lead to activation of EGFR degradation after cbl docking to this site

Conclusion: The results of this study show that ROS generation is essential for sanguinarine-induced apoptosis in H1975 cells and required at the early stage of the apoptosis.

### SUMMARY OF RESULTS IN EXAMPLES 1 TO 7

This is the first study demonstrating that sanguinarine exerts remarkable cytotoxic effect on killing Gefitinib-resistant NSCLC cells. It is also the first report showing that sanguinarine can effectively trigger degradation of EGFR. The results show that sanguinarine has potency of anticancer effect by potentiating EGFR degradation, MAPK (JNK and p38) activation, mitochondria disruption, and caspases activation. Taken together, these results indicate that sanguinarine could be used as a candidate agent against Gefitinib-resistant NSCLC patients, especially for the group of patients with EGFR^{L58R+T790M} mutation which represents 49% of all Gefitinib resistance cases.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### REFERENCES

1. Jemal, A., et al., Cancer statistics, 2010. CA: a cancer journal for clinicians, 2010. 60(5): p. 277-300.
2. Chang, A., Chemotherapy, chemoresistance and the changing treatment landscape for NSCLC. Lung cancer, 2011. 71(1): p. 3-10.
3. Tam, I. Y., et al., Double EGFR mutants containing rare EGFR mutant types show reduced in vitro response to gefitinib compared with common activating missense mutations. Molecular cancer therapeutics, 2009. 8(8): p. 2142-51.
4. Leung, E. L., et al., SRC promotes survival and invasion of lung cancers with epidermal growth factor receptor abnormalities and is a potential candidate for molecular-targeted therapy. Molecular cancer research: MCR, 2009. 7(6): p. 923-32.
5. Wong, D. W., et al., The EML4-ALK fusion gene is involved in various histologic types of lung cancers from nonsmokers with wild-type EGFR and KRAS. Cancer, 2009. 115(8): p. 1723-33.
6. Jun, H. J., et al., The oncogenic lung cancer fusion kinase CD74-ROS activates a novel invasiveness pathway through E-Syt1 phosphorylation. Cancer research, 2012. 72(15): p. 3764-74.
7. Ono, M. and M. Kuwano, Molecular mechanisms of epidermal growth factor receptor (EGFR) activation and response to gefitinib and other EGFR-targeting drugs. Clinical cancer research: an official journal of the American Association for Cancer Research, 2006. 12(24): p. 7242-51.
8. Sequist, L.V., et al., Genotypic and histological evolution of lung cancers acquiring resistance to EGFR inhibitors. Science translational medicine, 2011. 3(75): p. 75ra26.
9. Beuria, T. K., M. K. Santra, and D. Panda, Sanguinarine blocks cytokinesis in bacteria by inhibiting FtsZ assembly and bundling. Biochemistry, 2005. 44(50): p. 16584-93.
10. Obiang-Obounou, B. W., et al., The mechanism of action of sanguinarine against methicillin-resistant Staphylococcus aureus. The Journal of toxicological sciences, 2011. 36(3): p. 277-83.
11. Pencikova, K., et al., Investigation of sanguinarine and chelerythrine effects on LPS-induced inflammatory gene expression in THP-1 cell line. Phytomedicine: international journal of phytotherapy and phytopharmacology, 2012. 19(10): p. 890-5.
12. Niu, X., et al., The anti-inflammatory effects of sanguinarine and its modulation of inflammatory mediators from peritoneal macrophages. European journal of pharmacology, 2012. 689(1-3): p. 262-9.
13. Vrba, J., E. Orolinova, and J. Ulrichova, Induction of heme oxygenase-1 by Macleaya cordata extract and its constituent sanguinarine in RAW264.7 cells. Fitoterapia, 2012. 83(2): p. 329-35.
14. Vrba, J., et al., Sanguinarine is a potent inhibitor of oxidative burst in DMSO-differentiated HL-60 cells by a non-redox mechanism. Chemico-biological interactions, 2004. 147(1): p. 35-47.
15. Bai, L. P., et al., Site-specific binding of chelerythrine and sanguinarine to single pyrimidine bulges in hairpin DNA. Analytical and bioanalytical chemistry, 2008. 392(4): p. 709-16.

What is claimed is:

1. A method of preventing and treating Gefitinib-resistant non-small-cell lung cancer, comprising administering an effective amount of an isoquinoline alkaloid.

2. The method of claim 1, wherein said isoquinoline alkaloid is sanguinarine.

3. The method of claim 1 wherein said Gefitinib-resistant non-small-cell lung cancer is induced by EGFR^{L858R+T790M} mutation.

4. A pharmaceutical composition comprising an isoquinoline alkaloid admixed with a pharmaceutical carrier for treating Gefitinib-resistant non-small-cell lung cancer.

5. The pharmaceutical composition of claim 4, wherein said isoquinoline alkaloid is sanguinarine.

6. The pharmaceutical composition of claim 4 wherein said Gefitinib-resistant non-small-cell lung cancer is induced by EGFR^{L858R+T790M} mutation.

7. A dietary supplement comprising an isoquinoline alkaloid admixed with a pharmaceutical carrier for treating Gefitinib-resistant non-small-cell lung cancer.

8. The dietary supplement of claim 7, wherein said isoquinoline alkaloid is sanguinarine.
